(19) United States
(12) Patent Application Publication
(10) Pub. No.: US 2006/0257488 A1
(43) Pub. Date: Nov. 16, 2006

(54) Injectable hydrogels and methods of making and using same

(75) Inventor: William G. Hubbard, Burlington, WI (US)

Correspondence Address: MICHAEL BEST & FRIEDRICH, LLP, 100 E WISCONSIN AVENUE, MILWAUKEE, WI, 53202, US

(73) Assignee: Cytophil, Inc., Burlington, WI (US)

(21) Appl. No.: 11/431,276

(22) Filed: May 10, 2006

Related U.S. Application Data
(60) Provisional application No. 60/679,523, filed on May 10, 2005.

(51) Int. Cl. A61K 38/26; A61K 9/14

(52) U.S. Cl. 424/486; 524/425; 524/431; 524/423; 524/502; 524/556; 514/12

(57) ABSTRACT

A biocompatible hydrogel and method for augmenting soft and hard tissue, wherein the hydrogel comprises at least one gel former and the hydrogel is used to augment tissue when introduced into a desired tissue site. The hydrogel may comprise at least one of a carbomer, a poloxamer and a combination thereof. The hydrogel may have a yield strength of about 300 gm/cm-sec to about 12,000 gm/cm-sec and require less than about 10 lbf to extrude the hydrogel from a 1 cc syringe having a 25 gauge ½″ length needle at a rate of 2 inches per minute.

### CROSS-REFERENCE TO RELATED APPLICATIONS

This application claims priority under 35 U.S.C. §119(e) to U.S. Provisional Patent Application No. 60/679,523 filed May 10, 2005, the entire content of which is hereby incorporated by reference.

### BACKGROUND

Hydrogels made from gel formers have been previously investigated or used for delivery of drugs or other injectable materials. Some existing hydrogels have been shown to be degraded and removed from the placement site within a few weeks. In addition, some existing hydrogels are not highly cohesive and disperse after injection. Furthermore, some hydrogels have been shown to degrade relatively quickly in vivo. Accordingly, when such hydrogels include particles suspended therein for soft and/or hard tissue augmentation, the relatively fast degradation of the hydrogel leads to inadequate results.

### SUMMARY

In one embodiment, the invention provides a biocompatible hydrogel for augmenting tissue, the hydrogel comprising at least one of a carbomer, a poloxamer and a combination thereof, the hydrogel augmenting tissue when introduced into a desired tissue site.

In another embodiment the invention provides a hydrogel comprising at least one gel former, wherein the hydrogel has a yield strength of about 300 gm/cm-sec to about 12,000 gm/cm-sec and requires less than about 10 lbf to extrude the hydrogel from a 1 cc syringe having a 25 gauge ½″ length needle at a rate of 2 inches per minute.

In a further embodiment the invention provides a method for augmenting soft or hard tissue, the method comprising introducing at a desired soft or hard tissue site a hydrogel comprising at least one of a carbomer, a poloxamer and a combination thereof to augment the soft or hard tissue site.

Other aspects of the invention will become apparent by consideration of the detailed description and accompanying drawings.

### DETAILED DESCRIPTION

Before any embodiments of the invention are explained in detail, it is to be understood that the invention is not limited in its application to the details of construction and the arrangement of components set forth in the following description. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

U.S. Pat. Nos. 5,922,025, 6,432,437, 6,537,574 and 6,558,612 issued to William G. Hubbard, and Misiek, D. J., et al., “Soft Tissue Responses to Hydroxylapatite Particles of Different Shapes,” J. Oral Maxillofacial Surgery, 42:150-160, 1984 are incorporated herein by reference.

As used herein, the term “injectable” refers generally to being placed beneath the outer layer of skin, or deeper, using at least one of a needle, a fine cannula, and the like.

As used herein, the term “gel former” refers generally to a polymer (including homopolymers and copolymers), or combination thereof, that can be used to form a hydrogel.

The present invention generally relates to hydrogels (e.g., synthetic hydrogels) with specific characteristics that, alone or combination with other gel formers and/or other additives, can provide improved injection characteristics, improved corrections and improved durability of corrections for soft and/or hard tissue augmentation in vivo. In some embodiments of the present invention, the hydrogel includes particles suspended therein (the combination of the hydrogel and particles is sometimes referred to herein as a “filler” or a “filler formulation”). The hydrogels of the present invention, with or without particles suspended therein, can be used in a variety of applications, including, but not limited to, at least one of plastic surgery/cosmetic applications, including facial folds, wrinkles, etc.; ear, nose and throat (ENT) applications, including vocal fold medialization, glottic insufficiency, sinus filling, snoring, sleep apnea, etc.; sphincter augmentations, including urinary, anal, and gastrointestinal sphincter augmentations, etc.; dental applications, including extraction sites, dental implants, ridge augmentation, papilla plumping, etc.; orthopedic applications, including bone void filling, spinal fusion, oral/maxillofacial filling, cranial filling, use with orthopedic implants to improve bone bonding and infiltration, etc.; tissue marking applications, such as for location or identification; drug delivery, alone or in combination with any of the above applications; and, combinations thereof. In some embodiments, hydrogels of the present invention may be useful as a short-term implant (e.g., six months or less), such as for vocal fold injections as a temporary correction of vocal fold insufficiency (e.g., following a stroke where there may be a reversible loss of function). Those skilled in the art will be familiar with these and other tissue augmentations, in which the hydrogels of the present invention can be applied.

Hydrogels of the present invention can be formed from gel formers that are highly cross-linked and which may be of sufficiently high molecular weight to form three-dimensional matrices. Examples of gel formers that can be used with the present invention include polyethylene oxide; polypropylene oxide; polyoxyethylene-polyoxypropylene block copolymers such as, but not limited to, those designated by the CTFA names Poloxamer 407 (CAS 9003-11-6, molecular weight 9,840-14,600 g/mol; available from BASF as Lutrol® F127) and Poloxamer 188 (CAS 9003-11-6, molecular weight 7680-9510 g/mol; available from BASF as Lutrol® F68); acrylic polymers such as, but not limited to, carbomers (e.g., Carbopol® 974P NF, CAS 9003-01-6; available from Noveon); polyvinylpyrrolidones (e.g., polyvinylpyrrolidones available from BASF and International Specialty Products (ISP)); polyethylene glycols (PEGs) (e.g., PEGs available from Nektar); gelatin (e.g., gelatin available from Gelita), polyvinyl alcohols (PVA); polyhydroxyethyl methacrylate (poly-HEMA or PHEMA); cellulose; alginate; hyaluronic acid; chitin and combinations thereof.

The hydrogels of the present invention can be formed from individual gel formers or as a combination of gel formers. For example, a poloxamer and another gel former (e.g., gelatin, cellulose and/or a Carbopol) may be used in combination to attain the desired characteristics. In addition, various forms of the same gel former (e.g., Poloxamer 188 and Poloxamer 407) can be combined to attain the desired characteristics.

The hydrogels of the present invention may be cross-linked before or after hydrogel formation, either chemically or by other means, such as ultraviolet radiation. Some materials are linear and others are more highly cross-linked. The degree of cross-linking can vary depending on the gel former used, and the degree of cross-linking can be a material-specific characteristic.

In some embodiments of the present invention, the gel former(s) used to create the hydrogels have a molecular weight of at least about 5,000 g/mol. In some embodiments, the higher the molecular weight of the gel former, the less gel former required to attain the desired characteristics. As a result, in some embodiments, the gel former(s) have a molecular weight of at least about 1 million grams/mole.

As mentioned above, the hydrogels of the present invention can include particles suspended therein, and can be used to aid in the injection of these particles. The particles used with the present invention can be synthetic or natural in origin. For example, the particles can include synthetic calcium hydroxylapatite or calcium phosphate obtained from a bovine source. In some embodiments, the particles are relatively insoluble (e.g., polymethylmethacrylate). In some embodiments, the particles are at least partially soluble (e.g., polylactic acid). Particles with a narrow or wide distribution of sizes can be used, or select sizes can be combined. A hydrogel of the present invention can include a single type of particle or a combination of particles suspended therein. In some embodiments, a particulate ceramic material is homogeneously suspended in the gel prior to and during introduction of the biocompatible composition to the desired site.

Particles used with the present invention can include, but are not limited to, calcium phosphate, calcium hydroxylapatite, alpha tricalcium phosphate, beta tricalcium phosphate, calcium pyrophosphate, tetracalcium phosphate, octacalcium phosphate, calcium carbonate, fluorapatite, alumina, zirconia, carbon, polymethylmethacrylate, polyglycolic acid, polylactic acid, ceramic, glass, metal, polymers, and combinations thereof. The particles can be used for soft tissue and/or hard tissue augmentation.

The particles of the present invention may be smooth rounded, substantially spherical particles having a diameter that can be characterized by, for example, sieve analysis or microscopic measurement. The term “substantially spherical” as used herein means that some of the present particles may be spheres, while others are sphere-like in their shape, i.e., they are spheroidal. In one embodiment, the particles are spheroidal, rather than spherical. In another embodiment, the particles are irregular particles, or rounded or smooth rounded irregular particles. The term “irregular particles” as used herein refers to particles that are produced by fracturing or breaking larger particles. The terms “rounded” or “smooth rounded” as used herein refer to the irregular particles that have corners or angular edges, even though they may be polished smooth by finishing processes. Examples of such particles and methods for making the same are described in U.S. Pat. No. 6,432,437 and in Misiek, D. J., et al., “Soft Tissue Responses to Hydroxylapatite Particles of Different Shapes,” J. Oral Maxillofacial Surgery, 42:150-160, 1984, each of which is hereby fully incorporated by reference. The particles may also be substantially non-spherical having dimensions that can also be characterized by sieve analysis or by microscopic measurement. Non-spherical particles include, but are not limited to, particles having textured porous surfaces or openings, porous particles, particles having jagged, irregular shapes, and particles having straight edges.

The particles used with the present invention can be chosen based on their size. A sieve analysis can be used to identify and/or isolate particles of the desired size. In some embodiments of the present invention, the particles have a size of less than about 1000 microns. In additional embodiments, the particles range in size from about 15 microns to about 1000 microns. In further embodiments, the particles range in size from about 25 microns to about 420 microns. In yet further embodiments of the present invention, the particles range in size from about 25 microns to about 50 microns. Particles in this size range can be suitable for injection through fine needles. In other embodiments, the particles range in size from about 250 microns to about 420 microns. In some embodiments, the particles include a combination of sizes ranging from about 25 microns to about 50 microns and from about 250 microns to about 420 microns.

The rheological characteristics of a hydrogel can be defined by its viscosity, extrusion properties, and elasticity. When a force, or stress, is applied to a hydrogel, the shape of the hydrogel will undergo deformation. Viscosity is a measure of the hydrogel's ability to resist deformation. The hydrogels of the present invention generally have a viscosity (e.g., as measure by a viscometer, such as a Brookfield viscometer) of at least about 25,000 centipoise (cp).

In cases where the viscosity of a hydrogel decreases as an applied force increases, the hydrogel undergoes shear thinning and is said to be thixotropic. Shear thinning is a useful property for hydrogels that are introduced to a site by injection. As pressure is placed on the plunger of a syringe, the viscosity of the hydrogel decreases, making it possible to push the viscous hydrogel through a fine needle with a pressure that can readily by applied by hand. Hydrogels in the present invention typically require less than about 10 lbf, particularly less than about 8 lbf, and more particularly less than about 6 lbf to extrude a hydrogel through a 1 cc syringe having a 25 gauge ½ inch needle at an extrusion rate of 2 inches per minute.

Elasticity is the ability of the hydrogel to hold particles in suspension (in the case of a filler) and the ability of the hydrogel to return to its original shape after a force is applied.

Yield strength provides one method for assessing the elasticity of a hydrogel. Yield strength, or elasticity limit, is the amount of force that needs to be applied so that the original shape of the hydrogel will be permanently changed. In general, the hydrogels of the present invention have yield strength values from about 300 gm/cm-sec to about 12,000 gm/cm-sec and more particularly from about 500 gm/cm-sec to about 12,000. The desired yield strength, however, will vary somewhat with the nature of the site to which the hydrogel will be introduced. Hydrogels of the present invention for use in intradermal applications may have yield strength values from about 300 gm/cm-sec to about 5000 gm/cm-sec, and more particularly from about 300 gm/cm-sec to about 1,500 gm/cm-sec. Hydrogels for use in subdermal applications may have yield strength values from about 1,500 gm/cm-sec to about 7,500 gm/cm-sec, and more particularly from about 3,500 gm/cm-sec to about 7,500 gm/cm-sec. Hydrogels for use in bone applications may have yield strength values from about 3,500 gm/cm-sec to about 12,000 gm/cm-sec, and more particularly from about 5,000 gm/cm-sec to about 12,000 gm/cm-sec. Hydrogels encased within a suitable shell prior to implantation typically have yield strength values from about 1,500 gm/cm-sec to about 10,000 gm/cm-sec, and more particularly from about 3,500 gm/cm-sec to about 7,500 gm/cm-sec.

Elasticity of fillers can be measured by determining the degree to which particles separate from the hydrogel when the filler is subjected to centrifugal force. Particles will undergo separations from the hydrogel of less than about 5 mm, particularly less than about 3 mm, and more particularly less than about 2 mm when a 2.5 gram sample of the filler is centrifuged at 500 g's for 5 minutes.

As noted above, hydrogels of the present invention may be thixotropic (i.e., have shear-rate dependent viscosities), and/or exhibit yield strength, and/or elasticity. Additionally, the hydrogels may have a temperature-dependent (i.e., positive and/or negative) viscosity. As a result, the viscosity of the hydrogels can be measurement dependent, and different types of measurements may produce different viscosity measurements. In some embodiments, an oscillating rheometer can be used to measure the viscosity and elasticity of the hydrogels. In some embodiments, the bonding mechanisms and the cross-linking characteristics of a hydrogel can give the hydrogel its thixotropic properties. In the case of fillers, the Theological properties of the hydrogels are tailored to ensure particles are adequately suspended in the hydrogel during storage, so that the blend does not require mixing prior to injection, and during injection or implantation.

Some of the hydrogels of the present invention, such as hydrogels formed from a poloxamer, include a reversible thermal transition property, such that there can be a significant increase in the viscosity and elasticity with a relatively short temperature increase. For example, if the hydrogel is relatively thin at room temperature but becomes significantly thicker at body temperature, the pressure required to inject the hydrogel can be significantly decreased, while still providing augmentation when warmed to body temperature. Such properties can be especially applicable in cosmetic applications (e.g., smoothing fine lines and wrinkles in the face, treatment of other fine defects in the facial skin, and intradermal corrections), and applications that provide bulking of an individual implant site (e.g., applications that reduce the appearance of deep lines such as nasolabial folds, assist with treatment of urinary or fecal incontinence, or assist with treatment of gastroesophageal bulking).

The thermal transition temperature at which the viscosity and elasticity of the hydrogel increases or decreases depends on the type of poloxamer gel former, the amount of gel former, as well as the addition of any additives. In some embodiments of the present invention, the hydrogel includes a thermal transition temperature in the range of about 15° C. to about 37° C.

Some of the hydrogels of the present invention, such as hydrogels formed from carbomers, have significant yield strength, as well as being viscous and thixotropic. For example, the hydrogel has sufficient yield strength to provide soft tissue correction when injected (with or without particles) and the capability to hold particles in suspension prior to and after injection. In addition, these characteristics can be control by the extent of the neutralization (either prior to or after injection), the amount of gel former, and/or the addition of other additives. These combinations of characteristics allow ease of injection and placement as well as improved correction. In some embodiments, increasing the pH of the hydrogel (by addition of NaOH for example) by about 1 pH unit more than triples the yield strength of the hydrogel. In other embodiments, increasing the pH of the hydrogel by about 1 pH unit more than quintuples the yield strength.

In some embodiments of the present invention, the hydrogel includes other additives or gel formers to provide adequate rheological characteristics. For example, in some embodiments, the rheological characteristics are optimized to provide adequate particle suspension and injection. As used herein, the term “additive” refers to any substance or material that is added to the hydrogel to obtain a desired effect or property, including, without limitation, stability, absorption/resorption rate, pH, viscosity, elasticity or other desired rheological characteristics. Additives can include, but are not limited to, at least one of glycerin, polyethylene glycol (PEG), propylene glycol, one or more surfactants (e.g., Tween), any other additives commonly used in injectable drugs, and combinations thereof. These materials can be acquired from a variety of chemical suppliers, including VWR International, J. T. Baker, EMD, Baxter and Malinckrodt. In some embodiments, the additive can be used in a concentration from as low as a few percent up to almost about 100% of the hydrogel formulation, such as with lower molecular weight additives (e.g., glycerin, PEG, etc.).

In some embodiments of the present invention, the hydrogel includes one or more drugs, including, without limitation, at least one of lidocaine (i.e., for pain reduction), epinephrine (i.e., for reduction of swelling and bruising), and combinations thereof. In some embodiments of the present invention, the hydrogel includes one or more growth factors, including, without limitation, P15 human growth hormone, which can be used to promote healing and cellular infiltration, and βPDGF. Lidocaine, epinephrine, P15 human growth hormone and βPDGF are given by way of example only. It should be understood that a vast variety of drugs and growth factors can be included without departing from the spirit and scope of the present invention. Effective amounts of drugs and/or growth factors will be readily ascertainable to those having skill in the art.

In some embodiments, the hydrogel is formed from one or more gel formers (and any additives, drugs or growth factors) and water. In other words, the base (or solvent) of the hydrogel is water in some embodiments. However, in other embodiments, the base is a combination of water and one or more tonicity agents including, without limitation, at least one of saline, sucrose, mannitol, phosphate buffers, other common additions used in aqueous formulations, and combinations thereof. These materials can be acquired from a variety of chemical suppliers, including VWR International, J. T. Baker, EMD, Baxter and Malinckrodt. By selecting the appropriate tonicity agent, a hydrogel may comprise a hypotonic solution, a hypertonic solution or an isotonic solution. In some embodiments, the solution of the gel former and the base used to form the hydrogel includes one or more pH modifiers, including, without limitation, at least one of an acid (e.g., hydrochloric acid), a base (e.g., sodium hydroxide), a pH-affecting additive (e.g., triethanolamine (TEA)), and combinations thereof to adjust the pH of the solution.

Hydrogels of the present invention can also be prepared using non-aqueous bases (or solvents), including, without limitation, at least one of glycerin, PEG, and combinations thereof. Some of the particles used with the present invention can be readily or partially soluble in aqueous hydrogels or water. As a result, non-aqueous bases can be used to form a hydrogel that will minimize dissolution of such particles. In addition, hydrogels formed from non-aqueous bases can include drugs that degrade in aqueous solutions or that are not stable in aqueous solutions.

Hydrogels of the present invention may include about 15% to about 30% by weight poloxamer, more particularly about 17.5% to about 25%, and even more particularly about 20% to about 25%. Hydrogels of the present invention may also include about 0.2% to about 4% by weight carbomer, more particularly about 0.5 to about 2.0%, and even more particularly about 0.7% to about 1.5%. Hydrogels of the present invention may further include about 70% to about 99.8% by weight base (or solvent), more particularly about 85% to about 99.5%, and even more particularly about 85% to about 99.2%. When particles are added to such hydrogels, the hydrogels may include about 15% to about 55% by volume particles, more particularly about 25% to about 50%, and even more particularly about 30% to about 45%. The hydrogels of the present invention may include about 3% to about 99% by weight additives, more particularly about 5% to about 25%, and even more particularly about 5% to about 15%. The base (or solvent) may include about 0.1% to about 20% by weight tonicity agents, more particularly about 0.5% to about 10%, and even more particularly about 0.9% to about 6%. The base (or solvent) may also include about 0.1% to about 10% by weight pH modifiers, more particularly about 0.2% to about 8%, and even more particularly about 0.2% to about 5%.

In some embodiments of the present invention, particles that are to be introduced by implantation or injection can be dry-coated with one or more gel formers, so that these particles, when implanted, can form a hydrogel and can become a cohesive implant. Such dry-coating of particles can be used in a variety of applications, including, without limitation, in dental implants or other boney (hard) tissue applications where direct application of the particles to the site of implant is preferred. In some embodiments, the particles are suspended within the hydrogel, and the hydrogel is dehydrated to form a strip or block of hydrogel and particles.

In other embodiments of the present invention, the hydrogel is encased within a suitable shell, and the hydrogel-filled shell is then implanted at the desired site. The shell may be made of a polymeric material such as, but not limited to, polyurethanes, ethylene-propylene diene monomers, ethylene-propylene rubbers, polyolefins, and silicone elastomers.

### EXAMPLES

Procedures for the rheological tests conducted on various samples are outlined below.

Extrusion

A sample of the hydrogel is placed in a 1 cc B-D syringe. The force required to extrude the hydrogel from the syringe having a specified gauge needle at a rate of 2 inches per minute is measured. Needles used in the analysis include: 25 gauge ⅝″; 27 gauge ½″, 30 gauge ½″, 20 gauge 1″, 21 gauge 1″, 22 gauge 1″ and 23 gauge 1″.

Yield Strength

Yield strength is determined using a slightly modified version of the suspended sphere method described by Cohen in Bulletin 12: Flow and Suspension Properties, Noveon, pp. 7-8. Spheres of varying density and varying size are suspended in a hydrogel. The location of each sphere within the hydrogel is marked on the container wall, and the hydrogel is stored for one week at a specific temperature. If no appreciable sphere movement is noted, it is considered suspended. The yield value of the hydrogel is associated with the largest, most dense sphere that remains suspended.

$$Minimum Yeild Value Needed to Suspend a Sphere = \\ \begin{matrix} = \frac{( \begin{matrix}Volume \times Density \\ of Sphere\end{matrix} - \begin{matrix}Volume \times Density \\ of Displace Gel\end{matrix} ) \times ( \begin{matrix}Acceleration Due \\ to Gravity\end{matrix} )}{Cross \text{-} sectional Area of Sphere} \\ = 4 / 3 R ( D - {D}_{*} ) g in gm \text{/} cm \text{-} {sec}^{2}\end{matrix}$$

$$where \text{:}$$

$$R = sphere radius ( cm )$$

$$D = sphere density ( gm \text{/} cc )$$

$${D}_{*} = gel density ( gm \text{/} cc )$$

$$G = gravity ( 980 cm \text{/} {sec}^{2} )$$

The following spheres are used in determining the yield strength: stainless steel −1″, ⅞″, ¾″, ⅝″, ½″, 7/16″, ⅜″, 11/32″, 5/16″, 9/32″, ¼″, 7/32″, 3/16″, 5/32″, ⅛″, 3/32″, 1/16″, 9/16″, 11/16″ and 15/16″; Teflon- 5/16″, ¼″, 3/16″ and ⅛″; and nylon −¾″, ½″, ⅜″ and ¼″. The true yield strength may be slightly higher (between the suspended sphere and the next larger sphere which began to settle).
Centrifuge Separation

A 2.5 microgram sample of a hydrogel comprising suspended particles is centrifuged at 500 g's for 5 minutes. The sample is then evaluated to determine the degree to which the particles separate from the gel. The separation is determined by the portion of the sample that the particles settled out of during the centrifuging that is now clear of the particles and is visible as clear gel. Gels with high suspension (yield strength) typically exhibit a separation of about 3 mm or less.

The following examples are meant to be illustrative and not limiting. Unless stated otherwise, concentrations are given in % by weight.

### Example 1

### Durable Filler Formulation

A filler according to the present invention comprising a blend of a hydrogel and smoothed, irregular particles of calcium hydroxylapatite (CaHA) having particle sizes ranging from about 25 microns to about 50 microns was prepared. The hydrogel formulation (% w/w) included: 20% Poloxamer 407 (available from BASF as Lutrol® F127), 20% polyethylene oxide 300 and 1% polyvinylpyrrolidone in phosphate-buffered saline (aqueous) solution (available from Baxter) having a pH of about 7. The filler formulation (% v/v) included: 60% hydrogel formulation and 40% CaHA particles. The hydrogel and the CaHA particles were blended using low shear mixing, or a simulation of low shear mixing, such as hand mixing (e.g., with a spatula), or mixing with a paddle-type mixer.

### Example 2

### Hydrogel Comprising Poloxamers

A hydrogel according to the present invention comprising the following hydrogel formulation (% w/w): 21% Poloxamer 407 and 15% Poloxamer 168 (available from BASF as Lutrol® F68) in phosphate buffered saline (aqueous) solution was prepared.

### Example 3

### Influence of Solvent on Carbomer Hydrogels

A mixture of 98.5% phosphate buffered saline solution (PBS) and 1.5% carbomer was prepared in the following manner: 3 grams of Carbopol® 974P NF polymer (available from Noveon) was added to 197 grams of water. The reagents were mixed together by slowly adding the Carbopol® 974P NF into the vortex created by stirring the water. The mixture was then blended with a paddle mixer at a medium speed for about 30 minutes to ensure dispersion of the Carbopol® 974P NF. The Carbopol® 974P NF solution was then partially neutralized with 7.5 cc of a 20% sodium hydroxide solution. The resulting mixture was a low viscosity gel (Gel 1) with a pH of 5.0.

A mixture of 99.25% sterile water for injection and 0.75% carbomer was prepared in the following manner: 1.5 grams of Carbopol® 974P NF was added to 198.5 grams of water in a vessel large enough to mix the entire batch. The reagents were mixed together by slowly adding the Carbopol® 974P NF into the vortex created by stirring the water. The mixture was then blended with a paddle mixer at a medium speed for about 30 minutes to ensure dispersion of the Carbopol® 974P NF. The Carbopol® 974P NF solution was then neutralized with 3 cc of a 20% sodium hydroxide solution. The resulting mixture was a high viscosity gel (Gel 2) with a pH of 7.0.

One set of 1 cc syringes was filled with Gel 1. Another set of 1 cc syringes was filled with Gel 2. The syringes were capped with a male Luer cap. The gel filled syringes were then placed into a Tyvek pouch and sterilized in an autoclave at 251° F. for 30 minutes.

0.25 cc samples of Gel 1, Gel 2 and a control were injected subcutaneously in the backs of two groups of rabbits. The control gel was made from a well-known sodium carboxymethylcellulose (NaCMC) gel comprising 3% NaCMC in a 15/85: glycerin/water solution (available from Bioform Medical as Radiesse™ Gel). One group of rabbits was terminated after 2 weeks and the other group was terminated after 4 weeks.

The implant sites were examined grossly, and histology was examined using microscopic examination. Gel 1, Gel 2 and the control gel samples yielded ‘non-significant’ gross reactions. The histological examinations revealed minimal tissue reaction in all three samples. All three samples were classified as non-irritants at both time periods. However, the NaCMC gel sample showed evidence of degradation at 2 weeks and considerable degradation at 4 weeks. The Gel 1 and Gel 2 samples were stable at both time periods and did not show any degradation at either time period.

The extrusion and yield strength values for Gel 1 and Gel 2 are summarized in Table 1. These results demonstrate the influence of the solvent vehicle on the physical properties of the resulting hydrogel.

TABLE 1

| | | Extrusion, | Extrusion, | | |
|---|---|---|---|---|---|
| | | lbf | lbf | | |
| | | 25 gauge | 27 | Extrusion, lbf | Yield |
| Solvent | | ⅝″ | gauge ½″ | 30 gauge ½″ | Strength |
| Vehicle | pH | needle | needle | needle | gm/cm-sec |
| PBS (Gel 1) | 5.0 | 0.9 | 1.1 | 2.0 | 3639 |
| Water (Gel 2) | 7.0 | 2.2 | 3.1 | 5.1 | 5095 |

The yield strength of these samples is significantly greater than the control using sodium carboxymethylcellulose gel and other commonly used tissue augmentation gels such as collagen and hyaluronic acid. For example, the yield strength of the sodium carboxymethylcellulose gel is 270 gm/cm-sec.

### Example 4

### Influence of Degree of Neutralization on Carbomer Hydrogels

The affect of the degree of neutralization on the physical properties of the resulting hydrogel was demonstrated by the following examples. A solution of 5.07% isotonic mannitol was prepared by adding 9.13 grams of mannitol to 180 grams of water and mixing with a paddle mixer at a low speed for 5 minutes. 21.10 grams of glycerin were added to the solution, and the solution was mixed for an additional 5 minutes. Then 2.13 grams of Carbopol® 974PNF were added to the solution and the solution was mixed for at least two hours at a slightly higher speed. A 20% NaOH solution was then added to the mixture in various amounts as shown in the table below. The solution gelled immediately upon addition of the 20% NaOH solution. The gel was then mixed for at least 5 additional minutes. The extrusion and yield strength values of the resulting hydrogels are summarized in Table 2.

TABLE 2

| | | Extrusion, | | | |
|---|---|---|---|---|---|
| | | lbf | | | |
| | | 25 | Extrusion, lbf | Extrusion, lbf | Yield |
| NaOH | | gauge ⅝″ | 27 gauge ½″ | 30 gauge ½″ | Strength |
| Addition | pH | needle | needle | needle | gm/cm-sec |
| none | 3.5 | 0.6 | 0.8 | 1.5 | 674 |
| 0.5 cc | 4.5 | 2.4 | 3.0 | 5.8 | 3639 |
| 1.0 | 4.5 | 3.1 | 4.0 | 7.8 | 6403 |
| 1.5 cc | 5.0 | 3.2 | 4.1 | 7.2 | 5823 |
| 2.0 cc | 5.5 | 3.3 | 4.5 | 7.5 | 7279 |
| 2.5 cc | 5.5 | 3.4 | 4.5 | 7.9 | 7279 |
| 3.0 cc | 6.0 | 3.4 | 4.6 | 7.4 | 6403 |
| 3.5 cc | 6.5 | 3.3 | 5.0 | 7.5 | 6403 |
| 4.0 cc | 7.0 | 3.7 | 4.8 | 8.2 | 6403 |
| 4.5 cc | 7.5 | 3.6 | 4.7 | 8.5 | 6403 |
| 5.0 cc | 8.0 | 3.4 | 4.4 | 7.6 | 7279 |

It can be observed that the solution readily changes to a thick, viscous thixotropic gel having significant yield strength with minimal amounts of a neutralizing agent.

The resulting mixtures have utility for different types of tissue augmentation. For example, a relatively thick gel with a higher yield strength has application for forming localized blebs or filling out deeper facial folds. While a thinner solution with a lower yield strength can be used to fill out fine wrinkles. The solution can be neutralized before it is injected or be neutralized in vivo by the physiological fluids according to the homeostatic mechanism.

### Example 5

### Influence of Carbomer Concentration on Carbomer Hydrogels

Hydrogels were synthesized using various concentrations of carbomer in water with and without neutralization. A hydrogel without neutralization was prepared by adding 1.0 g (0.5%) Carbopol® 974P NF to 199.5 grams of water in a vessel large enough to mix the entire batch. The reagents were mixed together by slowly adding the Carbopol® 974P NF into the vortex created by stirring the water. The mixture was then blended with a paddle mixer at a medium speed for about 30 minutes to ensure dispersion of the Carbopol® 974P NF. Additional samples were prepared by substituting the following amounts of Carbopol® 974P NF in the above procedure: 2.0 g (1.0%), 3.0 g (1.5%), 4.0 g (2.0%) and 8.1 g (4.0%). The extrusion values for the resulting gels are summarized in Table 3.

The hydrogels with neutralization were prepared according to the non-neutralized procedure above with the following additional step. After mixing at medium speed for about 30 minutes, a 20% NaOH solution was added stepwise with mixing until the pH of the mixture was about 6.5 to about 7.5. The extrusion and yield strength values for the resulting neutralized gels are summarized in Table 4.

TABLE 3
(As Mixed)

| | Extrusion, lbf | Extrusion, lbf | Extrusion, lbf |
|---|---|---|---|
| | 25 gauge ⅝″ | 27 gauge ½″ | 30 gauge ½″ |
| Carbomer % | needle | needle | needle |
| 0.5% | 0.3 | 0.4 | 0.9 |
| 1.0% | 0.6 | 0.6 | 1.2 |
| 1.5% | 0.7 | 1.0 | 1.8 |
| 2.0% | 1.0 | 1.6 | 3.4 |
| 4.0% | 2.5 | 4.5 | 11.0 |

TABLE 4
(Neutralized)

| | | | | Yield |
|---|---|---|---|---|
| | Extrusion, lbf | Extrusion, lbf | Extrusion, lbf | Strength |
| Neutralized | 25 gauge ⅝″ | 27 gauge ½″ | 30 gauge ½″ | gm/cm- |
| Carbomer % | needle | needle | needle | sec |
| 0.5% | 2.4 | 2.6 | 4.7 | 5823 |
| 1.0% | 3.5 | 3.7 | 7.1 | 6403 |
| 1.5% | 4.0 | 5.2 | 10.4 | 6403 |
| 2.0% | 4.0 | 5.8 | 11.9 | 8735 |
| 4.0% | 7.35 | 10.0 | >15 | >11,646 |

### Example 6

### Influence of Excipients on Carbomer Hydrogels

Carbomer hydrogels containing a variety of excipients were synthesized with and without neutralization. A hydrogel without neutralization was prepared from an aqueous solution comprising 15% PEG and 1% carbomer. The PEG was added to water and mixed with a paddle mixer at low speed for 5 minutes. Then Carbopol® 974P NF was added to the solution, and the solution was mixed for at least two hours at a slightly higher speed to produce the resulting gel. Additional samples were prepared by substituting 15% glycerin, 15% PPG and 5.07% mannitol for the 15% PEG above and adjusting the percentage of water accordingly. The extrusion and yield strength values for the resulting gels are summarized in Tables 5.

The hydrogels with neutralization were prepared according to the non-neutralized procedure above with the following additional step. After mixing for at least two hours at slightly higher speed, a 20% NaOH solution was added stepwise with mixing until the pH of the mixture was about 6.5 to 7.5. The extrusion and yield strength values for the resulting gels are summarized in Table 6.

TABLE 5
(As Mixed)

| | | Extrusion, | Extrusion, | | |
|---|---|---|---|---|---|
| | | lbf | lbf | | |
| | | 25 | 27 | Extrusion, lbf | Yield |
| | | gauge ⅝″ | gauge ½″ | 30 gauge ½″ | Strength |
| Excipient | % | needle | needle | needle | gm/cm-sec |
| PEG | 15% | 0.5 | 0.9 | 1.9 | 727 |
| Glycerin | 15% | 0.5 | 0.7 | 1.5 | 404 |
| PPG | 15% | 0.4 | 0.7 | 1.4 | 404 |
| Mannitol | 5.07% | 0.4 | 0.6 | 1.0 | 404 |

TABLE 6
(Neutralized)

| | | Extrusion, | Extrusion, | | |
|---|---|---|---|---|---|
| | | lbf | lbf | | |
| | | 25 | 27 | Extrusion, lbf | Yield |
| | | gauge ⅝″ | gauge ½″ | 30 gauge ½″ | Strength |
| Excipient | % | needle | needle | needle | gm/cm-sec |
| PEG | 15% | 2.9 | 4.3 | 8.5 | 5823 |
| Glycerin | 15% | 2.6 | 3.8 | 7.8 | 5823 |
| PPG | 15% | 3.0 | 4.0 | 8.2 | 6403 |
| Mannitol | 5.07% | 2.6 | 3.7 | 6.2 | 5095 |

### Example 7

### Influence of Excipient and Carbomer Concentrations on Hydrogels

Carbomer hydrogels, with and without neutralization, were prepared in which both the excipient and carbomer concentrations were varied. A hydrogel without neutralization was prepared by first adding sufficient mannitol to water to produce a 5.07% isotonic mannitol solution. The solution was mixed with a paddle mixer at low speed for 5 minutes, after which glycerin (5%, 10% or 15% by weight) was added to the solution. The solution was mixed for an additional 5 minutes. Then Carbopol® 974P NF (0.75%, 1.00% or 1.25% by weight) was added to the solution, and the solution was mixed for at least two hours at a slightly higher speed to produce the resulting gel. The extrusion values for the resulting gels are summarized in Tables 7.

The hydrogels with neutralization were prepared according to the non-neutralized procedure with the following additional step. After mixing for at least two hours at slightly higher speed, a 20% NaOH solution was added stepwise with mixing until the pH of the mixture was about 6.5 to 7.5. The yield strength values for the resulting neutralized gels are summarized in Table 8.

Neutralized and sterilized hydrogels were prepared according to the neutralized procedure above with the following additional step. The resulting gels were sterilized in an autoclave at 251° F. for 30 minutes. The extrusion values for the resulting sterilized and neutralized gels are summarized in Table 9.

TABLE 7
(As Mixed: Extrusion (lbf) through a 27 gauge ½″ needle)

| | | % Carbomer | | |
|---|---|---|---|---|
| | | 0.75% | 1.00% | 1.25% |
| Glycerin | 5% | 0.5 | 0.7 | 0.8 |
| | 10% | 0.6 | 0.7 | 1.0 |
| | 15% | 0.7 | 0.8 | 1.1 |

TABLE 8
(Neutralized: Yield Strength (gm/cm-sec))

| | | % Carbomer | | |
|---|---|---|---|---|
| | | 0.75% | 1.00% | 1.25% |
| Glycerin | 5% | 4367 | 4367 | 4367 |
| | 10% | 5823 | 6403 | 6403 |
| | 15% | 6403 | 6403 | 5823 |

TABLE 9
(Neutralized and Sterilized: Extrusion (lbf) through
a 27 gauge ½″ needle)

| | | % Carbomer | | |
|---|---|---|---|---|
| | | 0.75% | 1.00% | 1.25% |
| Glycerin | 5% | 2.8 | 3.6 | 4.3 |
| | 10% | 3.4 | 4.1 | 4.8 |
| | 15% | 3.2 | 4.4 | 5.0 |

### Example 8

### Influence of Sterilization on Carbomer Hydrogels

An important characteristic of implant materials is their ability to maintain their structural integrity during and after sterilization. Hydrogels were prepared according to the procedures in Example 7 using 1.00% Carbopol® 974P NF and 10% glycerin in 5.07% isotonic mannitol. The extrusion and yield strength values are summarized in Table 10. The data demonstrate the stability of the hydrogels after sterilization.

TABLE 10

| | | Extrusion, | Extrusion, | Extrusion, | |
|---|---|---|---|---|---|
| | | lbf | lbf | lbf | |
| | | 25 | 27 | 30 | Yield |
| | | gauge ⅝″ | gauge ½″ | gauge ½″ | Strength |
| Neutralized | Sterile | needle | needle | needle | gm/cm-sec |
| No | No | 0.5 | 0.7 | 1.5 | 727 |
| No | Yes | 0.6 | 0.8 | 1.5 | 727 |
| Yes | No | 3.3 | 4.2 | 7.6 | 6403 |
| Yes | Yes | 2.9 | 4.1 | 7.5 | 6403 |

### Example 9

### Filler Comprising Carbomer and Glass Particles

A carbomer gel formulation comprising 1.0% Carbopol® 974P NF and 5% glycerin in 5.07% isotonic mannitol was prepared according to the neutralization procedure in Example 7 with the following additional step. After neutralization, the gel was blended with 35% (by volume) glass particles (75 to 125 micron).

Similarly, a control was prepared using sodium carboxymethylcellulose (NaCMC) instead of the carbomer. The NaCMC gel was prepared by adding 15% glycerin to 85% water and mixing for 5 minutes with a paddle mixer at low speed. Then 3% of 7HF NaCMC (obtained from Aqualon Division of Hercules, Inc.) was added to the mixture and the resulting gel was mixed for a minimum of 5 minutes with a paddle mixer at low speed. The NaCMC gel was allowed to ‘swell’ or hydrate for a least 24 hours. The gel was then blended with 35% (by volume) glass particles (75 to 125 micron). The extrusion characteristics and ability of the gel to resist separation when centrifuged at 500 g's for 5 minutes are summarized in Table 11.

TABLE 11

| | Extrusion, | Extrusion, | Extrusion, | |
|---|---|---|---|---|
| | lbf | lbf | lbf | Centrifuge |
| | 20 gauge 1″ | 21 gauge 1″ | 22 gauge 1″ | 500 g's for |
| Gel Former | needle | needle | needle | 5 minutes |
| NaCMC Gel | 1.3 | 1.6 | 2.0 | Minimal |
| Carbomer Gel | 0.6 | 0.7 | 1.2 | Minimal |

The carbomer gel is a noticeably creamier composition than the NaCMC gel which is a manifestation of the high yield strength. The carbomer gel also has a lower extrusion force and is easier to inject.

### Example 10

### Filler Comprising Carbomer and Calcium Hydroxylapatite Particles

A carbomer gel formulation comprising 1.25% Carbopol® 974P NF and 5% glycerin in 5.07% isotonic mannitol is prepared according to the procedures in Example 7 with the following additional step. The gels were blended with 33% (by volume) calcium hydroxylapatite particles (25 to 45 micron). The extrusion characteristics and ability of the gel to resist separation when centrifuged at 500 g's for 5 minutes are summarized in Table 12.

TABLE 12

| | | Extrusion, lbf | Extrusion, lbf | Centrifuge |
|---|---|---|---|---|
| Gel | | 25 gauge ⅝″ | 27 gauge ½″ | 500 g's for 5 |
| Former | Neutralized | needle | needle | minutes |
| Carbomer | No | 1.27 | 1.62 | Separates |
| Carbomer | Yes | 5.3 | 7.0 | None |

With respect to Examples 3-10, other additives and excipients can be used to modify and control the design characteristics of the carbomer gels. For example, surfactants (such as Tween), polyvinyl pyrrolidone, various molecular weights of polyethylene glycol and other additives may also be used to modify and control gel characteristics.

### Example 11

### Non-Aqueous Gel

A non-aqueous gel was prepared by mixing 49.5 grams of glycerin with 0.5 grams of Carbopol® 974P NF. The mixture was neutralized using 1.0 grams of triethanolamine (TEA). TEA is just one alternative to NaOH that can be used to neutralize the carbomer. Other alternative bases include, but are not limited to, Ca(OH)₂. These components were blended together to form a viscous gel. Then 16.8 grams of this gel were blended with 33.3 grams of calcium hydroxylapatite particles. The resulting hydrogel formed a thick paste that could be extruded through a 21 gauge needle. The gel would be suitable for use with particles of calcium hydroxylapatite, as well as other particles, particularly if those particles are soluble or partially soluble in aqueous gels.

### Example 12

### Poloxamer Hydrogel

A mixture of 20% Poloxamer 407 (Lutrol® F127 from BASF) and 80% isotonic phosphate buffered saline (available from Baxter) was prepared in the following manner: 160 grams of cold (5° C.) phosphate buffered saline (PBS) water were transferred to a vessel large enough to mix the entire batch. The mixing vessel was placed in an ice bath and stirred with a magnetic stirrer. The Poloxamer 407 was slowly added to a vortex created in the PBS by agitating with the magnetic stirrer. This mixture was blended for 1.5 hours, and then the mixture was covered and stored at about 5° C. (refrigeration temperature).

The poloxamer mixture had very different Theological characteristics at room temperature and at refrigeration temperature. The mixture was a thin fluid at refrigeration temperature and a thick gel at room temperature. This resulted in very different extrusion characteristics and yield strengths as summarized in Table 13.

TABLE 13

| | | Extrusion, | Extrusion, | | |
|---|---|---|---|---|---|
| | | lbf | lbf | | |
| | | 23 gauge | 25 gauge | Extrusion, lbf | Yield |
| | | 1″ | ⅝″ | 27 gauge ½″ | Strength |
| Condition | Form | needle | needle | needle | gm/cm-sec |
| 5° C. | Liquid | 0.8 | 1.3 | 2.6 | <58 |
| 25° C. | Gel | 2.6 | 5.3 | 6.8 | 4367 |

The resulting mixtures have utility for different types of tissue augmentation. The mixture could be injected at low temperature and thicken, or become much more viscous, at body temperature. This allows easy injection or placement and fills a depression or defect when it warms to body temperature. For example, a relatively thick gel with a higher yield strength has application for forming localized blebs or filling out deeper facial folds. A thinner solution with a lower yield strength can be used to fill out fine wrinkles.

### Example 13

### In Vivo Testing

The hydrogels from Example 12 were filled into 1 cc syringes and the syringes were capped with a male Luer cap. The gel filled syringes were then placed into a Tyvek pouch and sterilized in an autoclave at 251° F. for 30 minutes.

0.25 cc samples of poloxamer and a control were injected subcutaneously in the backs of two groups of rabbits. The control gel was made from a well-known sodium carboxymethylcellulose gel comprising 3% NaCMC in a 15/85: glycerin/water solution (available from Bioform Medical as Radiesse™ Gel). One group of rabbits was terminated after 2 weeks and the other group was terminated after 4 weeks.

The implant sites were examined grossly, and histology was examined using microscopic examination. All samples yielded ‘non-significant’ gross reactions. The histological examinations revealed minimal tissue reaction in all three samples. All samples were classified as non-irritants at both time periods. However, the NaCMC gel sample showed evidence of degradation at 2 weeks and considerable degradation at 4 weeks. The poloxamer sample was stable at both time periods and did not show any degradation at either time period.

### Example 14

### Poloxamer Hydrogels at Various Concentrations

Additional hydrogel samples were prepared according the procedure in Example 12 using various concentrations of poloxamer (15%, 17.5%, 20%, 22.5%, 25% and 30%) and adjusting the concentration of isotonic buffered saline solution accordingly. The gel transition temperature and the yield strength for the resulting gels are summarized in Table 14.

TABLE 14

| | | Below Gel Transition | | | |
|---|---|---|---|---|---|
| | Gel | Extrusion, lbf | Yield | Above Gel Transition | |
| | Temp | 25 gauge ⅝″ | Strength | Extrusion, lbf | Yield Strength |
| Poloxamer % | ° C. | needle | gm/cm-sec | 25 gauge ⅝″ needle | gm/cm-sec |
| 15% | >45 | 0.8 | <58 | NA | NA |
| 17.5% | 24 | 0.6 | <58 | 1.9 | <58 |
| 20% | 20 | 0.3 | <58 | 2.4 | 4367 |
| 22.5% | 15 | 0.9 | <58 | 3.5 | 8027 |
| 25% | 13 | 1.8 | <58 | 4.7 | 10,190 |
| 30% | 4 | 9.1 | <58 | 9.5 | >11,646 |

### Example 15

### Mixed Blend Hydrogels

Additional hydrogel samples were prepared according the procedure in Example 12, with the exception that Poloxamer 188 was added in addition to Poloxamer 407. The concentration of isotonic buffered saline solution was adjusted accordingly. The concentrations of Poloxamer 407 and Poloxamer 188 in each sample, along with the extrusion and yield strength values for the resulting hydrogels, are summarized in Table 15.

TABLE 15

| | | | Below Gel Transition | | Above Gel Transition | |
|---|---|---|---|---|---|---|
| | | | Extrusion, | | Extrusion, | |
| | | Gel | lbf | | lbf | Yield |
| Poloxamer | Poloxamer | Temp | 25 gauge | Yield Strength | 25 gauge | Strength |
| 407% | 188% | ° C. | ⅝″ needle | gm/cm-sec | ⅝″ needle | gm/cm-sec |
| 21% | 10% | 27 | 2.9 | <58 | 3.0 | >11,646 |
| 20% | 15% | 24 | 4.9 | <58 | 3.6 | >11,646 |
| 17.5% | 17.5% | 29 | 2.4 | <58 | 4.6 | >11,646 |
| 20% | 20% | 19 | 5.6 | <58 | 15.0 | >11,646 |
| 15% | 20% | 36 | 2.2 | <58 | 5.6 | >11,646 |

The sample prepared with 17.5% Poloxamer 407 and 17.5% Poloxamer 188 was injected subcutaneously in the backs of rabbits according to the procedure in Example 13. This sample was found to be highly lubricous, which would make this example an excellent synovial fluid replacement.

### Example 16

### Excipients in Poloxamer Hydrogels

The properties of hydrogels can be further modified by the addition of excipients, such as propylene glycol, PEG, glycerin or surfactants. These formulations can also be made with isotonic solutions such as phosphate buffered saline, mannitol or other additives.

Hydrogels were prepared according to the procedure in Example 12. In the case of Example A, hydrogels were prepared from 20% Poloxamer 407, 5% Poloxamer 188, 20% propylene glycol and 55% isotonic phosphate buffered saline solution. In Example B, hydrogels were prepared from 22% Poloxamer 407, 20% propylene glycol and 58% isotonic phosphate buffered saline solution. The extrusion and yield strength values of the resulting gels are summarized in Table 16.

TABLE 16

| | | | Below Gel Transition | | Above Gel Transition | |
|---|---|---|---|---|---|---|
| | | | Extrusion, | Yield | Extrusion, | Yield |
| | | Gel | lbf | Strength | lbf | Strength |
| | | Temp | 25 gauge | gm/cm- | 25 gauge | gm/cm- |
| | Formulation | ° C. | ⅝″ needle | sec | ⅝″ needle | sec |
| Example A | Poloxamer 407 20% | 15 | 5.5 | <58 | 5.0 | 8735 |
| | Poloxamer 188 5% | | | | | |
| | Propylene Glycol 20% | | | | | |
| Example B | Poloxamer 407 22% | 4 | 5.2 | <58 | 7.2 | 8735 |
| | Propylene Glycol 20% | | | | | |

### Example 17

### Mixed Gels Comprising Carbomer and Poloxamer

A carbomer/poloxamer blend hydrogel was prepared in the following manner: 1.44 grams of Carbopol®® 974P NF were added to 199.5 grams of water at ambient temperature. The reagents were mixed together by slowly adding the Carbopol® 974P NF into the vortex created by stirring the water. The mixture was then blended with a paddle mixer at a medium speed for about 20 minutes to ensure dispersion of the Carbopol® 974P NF. The Carbopol® 974P NF solution was then neutralized by adding a 20% sodium hydroxide solution stepwise with mixing until the pH of the mixture was about 6.5 to about 7.5. Then 28.97 grams of Poloxamer 407 were blended into the mixture and the entire mixture was refrigerated. This carbomer/poloxamer blend produced a very thick gel with extrusion characteristics and yield strength values as summarized in Table 17. The gel was filled into 1 cc syringes, sterilized and injected subcutaneously into the backs of rabbits, according to the procedure set forth in Example 3. The carbomer/poloxamer blend was found to be as biocompatible and durable as the samples evaluated in Example 3.

TABLE 17

| | | Extrusion, | Extrusion, | | |
|---|---|---|---|---|---|
| | | lbf | lbf | Extrusion, lbf | Yield |
| | | 23 gauge | 25 gauge | 27 gauge | Strength |
| Condition | Form | 1″ needle | ⅝″ needle | ½″ needle | gm/cm-sec |
| 5° C. | Gel | 4.3 | 6.3 | 10.0 | 674 |
| 25° C. | Gel | 6.3 | 8.5 | 13.5 | 4003 |

### Example 18

### Poloxamer and Other Gel Formers

An example of a combination of gel formers was prepared by using both poloxamer and sodium carboxymethylcellulose. 166.29 grams of phosphate buffered saline solution (PBS) were transferred to a vessel large enough to mix the entire batch. The mixing vessel was placed in an ice bath and stirred. 30.05 grams of Poloxamer 407 were slowly added to a vortex created in the PBS by the stirring action. This mixture was blended for 45 minutes. The mixture (still in a cold water bath) was then stirred by a paddle mixer while 4.95 grams of sodium carboxymethylcellulose (NaCMC—Noveon Cekol 30,000) were added to the mixture. The mixture produced a viscous gel having extrusion characteristics and yield strengths as summarized in Table 18.

TABLE 18

| | | Extrusion, lbf | Extrusion, lbf | Extrusion, lbf | |
|---|---|---|---|---|---|
| | | 23 gauge | 25 gauge | 27 gauge | Yield Strength |
| Condition | Form | 1″ needle | ⅝″ needle | ½″ needle | gm/cm − sec |
| 5° C. | Gel | 1.9 | 3.6 | 10.0 | 270 |
| 25° C. | Gel | 4.6 | 6.3 | 9.7 | >11,646 |

It can readily be understood that by adjusting the relative amounts of poloxamer and gel former, such as shown in Examples 17 and 18, a formulation can be designed (with and without particles) to optimize at least one of injection characteristics, viscosity, yield strength and filling characteristics. Properties of the hydrogels can be further improved by the addition of additives (such as mannitol for tonicity) or other excipients (such as glycerin, propylene glycol, PEG or Tween).

It can readily be understood that by combining various gel formers with either thermal, pH or other gelling mechanisms, that formulations with and without particles can be designed to optimize at least one of injection characteristics, viscosity, yield strength and filling characteristics. For example, a carbomer solution (not neutralized) could be combined with NaCMC gel former that would have very high yield strength when neutralized in vivo.

It can readily be understood that by adjusting the relative amounts of carbomer and a gel former such as described above, a formulation can be designed (with and without particles) to optimize at least one of injection characteristics, viscosity, yield strength or filling characteristics. This could further be improved by the addition of additives (such as mannitol for tonicity) or other excipients (such as glycerin, propylene glycol, PEG or Tween).

Thus, the invention provides, among other things, hydrogels for utilization in soft and hard tissue augmentation. Various features and advantages of the invention are set forth in the following claims.

What is claimed is:

1. A biocompatible hydrogel for augmenting tissue, the hydrogel comprising at least one of a carbomer, a poloxamer and a combination thereof, the hydrogel augmenting tissue when introduced into a desired tissue site.

2. The hydrogel of claim 1, wherein the composition comprises a carbomer.

3. The hydrogel of claim 1, wherein the composition comprises a poloxamer.

4. The hydrogel of claim 1, further comprising particles suspended therein.

5. The hydrogel of claim 4, wherein the particles have a size ranging from about 15 microns to about 1000 microns.

6. The hydrogel of claim 4, wherein the particles comprise at least one of calcium phosphate, calcium hydroxylapatite, alpha tricalcium phosphate, beta tricalcium phosphate, calcium pyrophosphate, tetracalcium phosphate, octacalcium phosphate, calcium carbonate, fluorapatite, alumina, zirconia, carbon, polymethylmethacrylate, polyglycolic acid, polylactic acid, ceramic, glass, metal, polymers, and a combination thereof.

7. The hydrogel of claim 4, wherein the particles comprise calcium hydroxylapatite.

8. The hydrogel of claim 1, further comprising at least one drug.

9. The hydrogel of claim 8, wherein the drug comprises at least one of lidocaine, epinephrine and a combination thereof.

10. The hydrogel of claim 1, further comprising at least one growth factor.

11. The hydrogel of claim 10, wherein the growth factor comprises at least one of P15 human growth hormone, β FDGF and a combination thereof.

12. The hydrogel of claim 1, further comprising at least one of a hypotonic solution, a hypertonic solution and an isotonic solution.

13. The hydrogel of claim 1, further comprising at least one of glycerin, polyethylene glycol, propylene glycol, a surfactant and a combination thereof.

14. The hydrogel of claim 1, further comprising at least one additional gel former.

15. The hydrogel of claim 1, wherein the composition comprises both a carbomer and a poloxamer.

16. The hydrogel of claim 1, wherein the hydrogel has a yield strength of about 300 gm/cm-sec to about 12,000 gm/cm-sec and requires less than 10 lbf to extrude the hydrogel from a 1 cc syringe having a 25 gauge ½″ length needle at a rate of 2 inches per minute.

17. The hydrogel of claim 1, wherein the hydrogel is encapsulated by an elastomeric shell.

18. A kit comprising a syringe filled with the hydrogel of claim 1.

19. The hydrogel of claim 1, further comprising a particulate ceramic material homogeneously suspended in the gel prior to and during introduction of the biocompatible composition to the desired site.

20. The hydrogel of claim 1, wherein the hydrogel has a thermal transition temperature in the range of about 15° C. to about 37° C.

21. The hydrogel of claim 1, wherein increasing the pH of the hydrogel by about 1 pH unit more than triples the yield strength of the hydrogel.

22. A hydrogel comprising at least one gel former, wherein the hydrogel has a yield strength of about 300 gm/cm-sec to about 12,000 gm/cm-sec and requires less than about 10 lbf to extrude the hydrogel from a 1 cc syringe having a 25 gauge ½″ length needle at a rate of 2 inches per minute.

23. The hydrogel of claim 22, wherein the yield strength is about 1,500 gm/cm-sec to about 7,500 gm/cm-sec.

24. The hydrogel of claim 22, wherein the hydrogel requires less than about 8 lbf to extrude the hydrogel from a 1 cc syringe having a 25 gauge ½″ length needle at a rate of 2 inches per minute.

25. The hydrogel of claim 22, further comprising particles comprising at least one of calcium phosphate, calcium hydroxylapatite, alpha tricalcium phosphate, beta tricalcium phosphate, calcium pyrophosphate, tetracalcium phosphate, octacalcium phosphate, calcium carbonate, fluorapatite, alumina, zirconia, carbon, polymethylmethacrylate, polyglycolic acid, polylactic acid, ceramic, glass, metal, polymers, and a combination thereof.

26. The hydrogel of claim 25, wherein the particles within the hydrogel undergo less than about 5 mm of separation when a 2.5 gram sample of the hydrogel is centrifuged at 500 g's for 5 minutes.

27. The hydrogel of claim 25, wherein the particles within the hydrogel undergo less than about 3 mm separation when a 2.5 gram sample of the hydrogel is centrifuged at 500 g's for 5 minutes.

28. The hydrogel of claim 22, further comprising at least one of lidocaine, epinephrine and a combination thereof.

29. The hydrogel of claim 22, further comprising at least one growth factor.

30. The hydrogel of claim 22, further comprising at least one of a hypotonic solution, a hypertonic solution and an isotonic solution.

31. A method for augmenting soft or hard tissue, the method comprising introducing at a desired soft or hard tissue site a hydrogel comprising at least one of a carbomer, a poloxamer and a combination thereof to augment the soft or hard tissue site.

32. The method of claim 31, wherein the hydrogel is introduced to the desired site by at least one of injection and implantation.

33. The method of claim 31, further comprising placing the hydrogel in an elastomeric shell prior to introducing the hydrogel to the desired site.

34. The method of claim 31, wherein the hydrogel further comprises particles suspended therein.

35. The method of claim 34, wherein the particles have a size ranging from about 15 microns to about 1000 microns.

36. The method of claim 34, wherein the particles comprise at least one of calcium phosphate, calcium hydroxylapatite, alpha tricalcium phosphate, beta tricalcium phosphate, calcium pyrophosphate, tetracalcium phosphate, octacalcium phosphate, calcium carbonate, fluorapatite, alumina, zirconia, carbon, polymethylmethacrylate, polyglycolic acid, polylactic acid, ceramic, glass, metal, polymers, and a combination thereof.

37. The method of claim 34, wherein the particles comprise calcium hydroxylapatite.

38. The method of claim 31, wherein the hydrogel further comprises at least one drug.

39. The method of claim 38, wherein the drug comprises at least one of lidocaine, epinephrine and a combination thereof.

40. The method of claim 31, wherein the hydrogel further comprises at least one growth factor.

41. The method of claim 40, wherein the growth factor comprises at least one of P15 human growth hormone, β FDGF and a combination thereof.

42. The method of claim 31, wherein the hydrogel further comprises at least one of a hypotonic solution, a hypertonic solution and an isotonic solution.

43. The method of claim 31, wherein the hydrogel further comprises at least one of glycerin, polyethylene glycol, propylene glycol a surfactant and a combination thereof.

44. The method of claim 31, wherein the hydrogel further comprises at least one additional gel former.

45. The method of claim 31, wherein the hydrogel has a yield strength of about 300 gm/cm-sec to about 12,000 gm/cm-sec and requires less than about 10 lbf to extrude the hydrogel from a 1 cc syringe having a 25 gauge ½″ length needle at a rate of 2 inches per minute.

46. The method of claim 31, wherein the hydrogel is encapsulated by an elastomeric shell.

47. The method of claim 31, wherein the hydrogel further comprises a particulate ceramic material homogeneously suspended in the gel prior to and during introduction of the biocompatible composition to the desired site.

48. The method of claim 31, wherein the hydrogel has a thermal transition temperature in the range of about 15° C. to about 37° C.

49. The method of claim 31, wherein increasing the pH of the hydrogel by about 1 pH unit more than triples the yield strength of the hydrogel.

50. A method for augmenting soft or hard tissue, the method comprising introducing a particle dry-coated with at least one gel former into a desired soft or hard tissue site.
